(19) United States
(12) Patent Application Publication
(10) Pub. No.: US 2022/0133545 A1
(43) Pub. Date: May 5, 2022

(54) HIGHLY PORTABLE NEGATIVE-PRESSURE WOUND CLOSURESYSTEM

(71) Applicant: KCI Licensing, Inc., San Antonio, TX (US)

(72) Inventors: Kristine M. KIESWETTER, San Antonio, TX (US); Michael MARCROFT, San Antonio, TX (US); Christopher Brian LOCKE, Bournemouth (GB)

(21) Appl. No.: 17/576,636

(22) Filed: Jan. 14, 2022

Related U.S. Application Data
(63) Continuation of application No. 16/308,892, filed on Dec. 11, 2018.
(60) Provisional application No. 62/365,184, filed on Jul. 21, 2016.

(51) Int. Cl. A61F 13/00; A61F 13/02; A61M 1/00

(52) U.S. Cl. CPC A61F 13/00068; A61F 13/0216; A61M 1/90; A61M 2205/3592; A61M 2205/3561; A61M 2205/3584; A61M 2209/088; A61M 2205/3553

(57) ABSTRACT

An ultra-portable therapy system for treating a tissue site with negative pressure is disclosed. In some embodiments, the therapy system may include a wound dressing, a low-profile conduit, a therapy unit, and a communications device. Some embodiments may also include aspects of a therapy network, including communications networks and a remote monitoring center.

### CROSS-REFERENCE TO RELATED APPLICATIONS

This present invention is a Continuation of U.S. Patent Application No. 16/308,892, filed Dec. 11, 2018, which is a National Stage filing of International Patent Application No. PCT/US2017/041945, filed Jul. 13, 2017, which claims the benefit, under 35 USC 119(e), of the filing of U.S. Provisional Patent Application Ser. No. 62/365,184, entitled “Highly Portable Negative-Pressure Wound Closure System,” filed Jul. 21, 2016, which are incorporated herein by reference for all purposes.

### TECHNICAL FIELD

The invention set forth in the appended claims relates generally to tissue treatment systems. More particularly, but without limitation, the present disclosure relates to portable negative-pressure therapy systems.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” “sub-atmospheric pressure therapy,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for providing negative-pressure therapy are set forth in the appended claims. Illustrative embodiments are also provided to enable a person skilled in the art to make and use the claimed subject matter.

In some embodiments, a system for treating a tissue site may include an absorbent wound dressing, a fluid conduit, a therapy unit comprising a pneumatic pump, and a communications device. The communications device may be configured to transmit operational data of the therapy unit and configured to wirelessly communicate with a remote device.

In other example embodiments, a method for treating a tissue site may include applying an absorbent dressing to the tissue site, fluidly connecting the absorbent dressing to a therapy unit, and activating the therapy unit. The therapy unit may include a pneumatic pump and a communications device. Activating the therapy unit may provide a reduced pressure to the absorbent dressing and transmit usage data to a remote electronic device.

In yet other example embodiments, a system for treating a tissue site may include a therapy unit, a mobile device, and a network. The therapy unit may include a negative-pressure source and a processor configured to receive input information and to generate output information related to the delivery of negative pressure from the negative-pressure source to the tissue site. The mobile device may be adapted to receive and display information related to the delivery of negative pressure from the negative-pressure source to the tissue site and to collect instructions from a user related to operational parameters of the therapy unit. The network may be adapted to allow communications between the therapy unit and the mobile device.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a schematic block diagram of an example embodiment of a therapy network for treating a tissue site in accordance with this specification;

FIG. 2 is a perspective view illustrating additional details that may be associated with some example embodiments of a therapy system of FIG. 1;

FIG. 3 is an exploded view of the dressing of FIG. 2, depicted without a conduit interface and with an illustrative embodiment of a release liner for protecting the dressing prior to application at a tissue site;

FIG. 4 is a detail view taken at reference FIG. 4, depicted in FIG. 2, illustrating the dressing of FIG. 2 positioned proximate to tissue surrounding the tissue site;

FIG. 5 is a cut-away view of an illustrative embodiment of a conduit interface depicted in the dressing of FIG. 2;

FIG. 6 is a plan view of an illustrative embodiment of a low profile conduit assembly suitable for use with the system and the dressing of FIG. 2;

FIG. 7 is an exploded view of the low profile conduit assembly of FIG. 6;

FIG. 8 is a cross-section of an illustrative embodiment of a low-profile conduit in the low-profile conduit assembly of FIG. 6;

FIG. 9 is a screen shot of a graphical user interface (GUI) of an example embodiment of the mobile device of FIG. 1.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

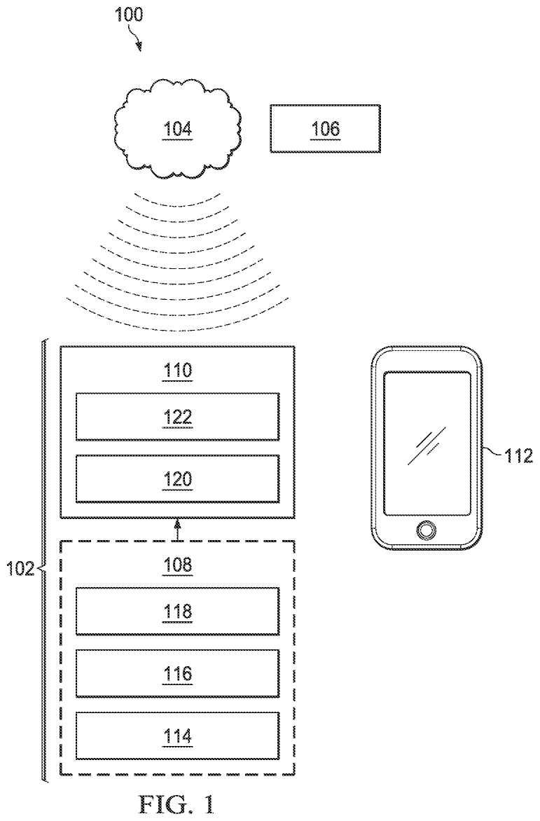

FIG. 1 is a schematic, block diagram, showing details of an illustrative embodiment of a therapy network 100 for treating a tissue site with negative pressure. The therapy network 100 may include therapy system 102, communication network(s) 104, and monitoring center 106. The therapy system 102 may be applied to a human patient, as well as used on other types of subjects. The therapy system 102 may include a dressing 108, a therapy unit 110, and a mobile telecommunications device 112. In some embodiments, the dressing 108 may include a tissue interface 114, a cover 116, and a conduit interface 118. In some embodiments, the therapy unit 110 may include a negative-pressure source 120 and a communication interface 122. The therapy system 102 may communicate with the monitoring center 106 through the communication network 104 and the communication interface 122.

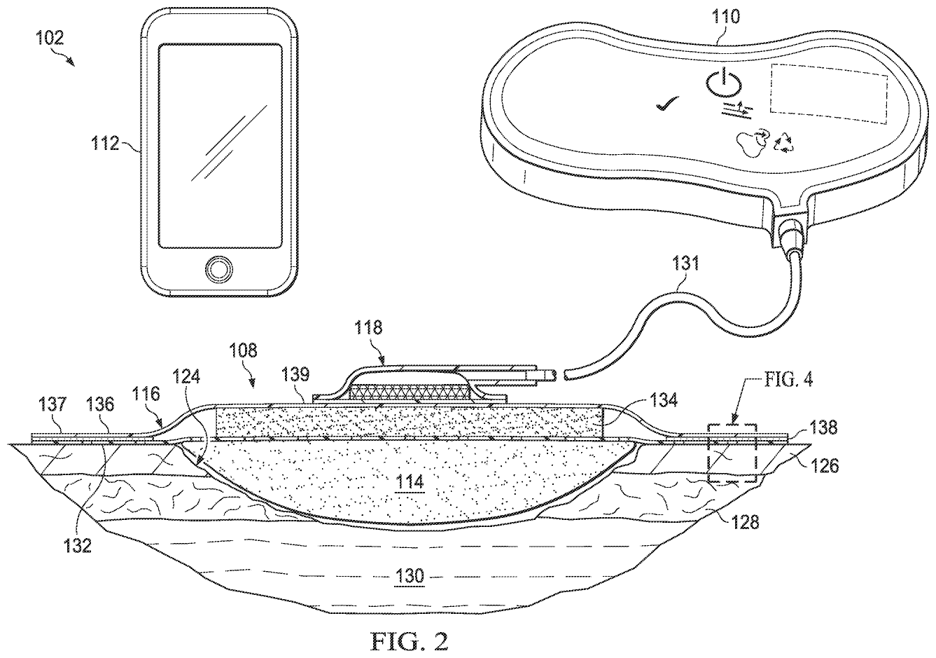

Referring now also to FIG. 2, additional details of the therapy system 102 of FIG. 1 for treating a tissue site 124 of a patient are shown. The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The tissue site 124 may extend through or otherwise involve an epidermis 126, a dermis 128, and a subcutaneous tissue 130. The tissue site 124 may be a sub-surface tissue site as depicted in FIG. 2, which may extend below the surface of the epidermis 126. The tissue site 124 may also be an incision, which may extend through the epidermis 126 and further into the dermis 128 and subcutaneous tissue 130. Further, the tissue site 124 may be a surface tissue site (not shown) that may predominantly reside on the surface of the epidermis 126. The therapy system 102 may provide therapy to, for example, the epidermis 126, the dermis 128, and the subcutaneous tissue 130, regardless of the positioning of the therapy system 102 or the type of tissue site. The therapy system 102 may also be utilized without limitation at other tissue sites.

Components of the therapy system 102 may be fluidly coupled to each other to provide a path for transferring fluids (i.e., liquid and/or gas) between the components. For example, components may be fluidly coupled through a fluid conductor, such as a tube. A “tube,” as used herein, broadly includes a tube, pipe, hose, conduit, or other structure with one or more lumina adapted to convey a fluid between two ends. Typically, a tube is an elongated, cylindrical structure with some flexibility, but the geometry and rigidity may vary. In some embodiments, components may also be coupled by virtue of physical proximity, being integral to a single structure, or being formed from the same piece of material. Moreover, some fluid conductors may be molded into or otherwise integrally combined with other components. Coupling may also include mechanical, thermal, electrical, or chemical coupling (such as a chemical bond) in some contexts. For example, a tube 131 may mechanically and fluidly couple the dressing 108 to the therapy unit 110 in some embodiments. In general, components of the therapy system 102 may be coupled directly or indirectly.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” generally refers to a pressure less than a local ambient pressure, such as the ambient pressure in a local environment external to a sealed therapeutic environment provided by the dressing 108. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −75 mm Hg (−9.9 kPa) and −300 mm Hg (−39.9 kPa).

A negative-pressure supply, such as the negative-pressure source 120 of therapy unit 110, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. A negative-pressure supply may also have one or more supply ports configured to facilitate coupling and de-coupling the negative-pressure supply to one or more distribution components.

Continuing with FIG. 2, the dressing 108 may be adapted to provide negative pressure from the negative-pressure source 120 of the therapy unit 110 to the tissue site 124, and to store fluid extracted from the tissue site 124. The dressing 108 of the therapy system 102 may include an optional tissue interface, such as a tissue interface 114. The tissue interface 114 is an optional component that may be omitted for different types of tissue sites or different types of therapy using negative pressure, such as, for example, epithelialization. If equipped, the tissue interface 114 may be adapted to be positioned proximate to or adjacent to the tissue site 124, such as, for example, by cutting or otherwise shaping the tissue interface 114 in any suitable manner to fit the tissue site 124. As described below, the tissue interface 114 may be adapted to be positioned in fluid communication with the tissue site 124 to distribute negative pressure to the tissue site 124. In some embodiments, the tissue interface 114 may be positioned in direct contact with the tissue site 124.

The tissue interface 114 can be generally adapted to contact a tissue site. The tissue interface 114 may be partially or fully in contact with the tissue site. If the tissue site is a wound, for example, the tissue interface 114 may partially or completely fill the wound, or may be placed over the wound. The tissue interface 114 may take many forms, and may have many sizes, shapes, or thicknesses depending on a variety of factors, such as the type of treatment being implemented or the nature and size of a tissue site. For example, the size and shape of the tissue interface 114 may be adapted to the contours of deep and irregular shaped tissue sites. Moreover, any or all of the surfaces of the tissue interface 114 may have projections or an uneven, course, or jagged profile that can induce strains and stresses on a tissue site, which can promote granulation at the tissue site.

In some embodiments, the tissue interface 114 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam, reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 114 may be a foam having pore sizes in a range of 400-600 microns. The tensile strength of the tissue interface 114 may also vary according to needs of a prescribed therapy. For example, the tensile strength of a foam may be increased for instillation of topical treatment solutions. In one non-limiting example, the tissue interface 114 may be an open-cell, reticulated polyurethane foam such as GranuFoam® dressing or VeraFlo® foam, both available from Kinetic Concepts, Inc. of San Antonio, Tex.

The tissue interface 114 may be either hydrophobic or hydrophilic. In an example in which the tissue interface 114 may be hydrophilic, the tissue interface 114 may also wick fluid away from a tissue site, while continuing to distribute negative pressure to the tissue site. The wicking properties of the tissue interface 114 may draw fluid away from a tissue site by capillary flow or other wicking mechanisms. An example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

The tissue interface 114 may further promote granulation at a tissue site when pressure within the sealed therapeutic environment is reduced. For example, any or all of the surfaces of the tissue interface 114 may have an uneven, coarse, or jagged profile that can induce microstrains and stresses at a tissue site if negative pressure is applied through the tissue interface 114.

In some embodiments, the tissue interface 114 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 114 may further serve as a scaffold for new cell-growth, or a scaffold material may be used in conjunction with the tissue interface 114 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft or xenograft materials, such as amniotic, adipose, dermal, liver, bladder, submucosal intestine materials, as well as others.

Continuing with FIG. 2, the cover 116 may be adapted to provide negative pressure from the negative-pressure source 120 of the therapy unit 110 to the tissue interface 114, and to store fluid extracted from the tissue site 124 through the tissue interface 114. In some embodiments, the cover 116 may include a base layer 132, an absorbent member 134, a sealing member 136, and an adhesive 138. Components of the cover 116 may be added or removed to suit a particular application.

The therapy unit 110 may further include a battery supply, which may include a rechargeable battery pack or disposable batteries. The therapy unit 110 may also include on-board control electronics, which in some embodiments, may include a simple on/off switch or button, as well as a simple indicator, such as a light, for providing status feedback. In some embodiments, the communication interface 122 may be integrated within the housing of the therapy unit 110. For example, the communication interface 122 may be incorporated as part of the electronic components contained within the therapy unit 110. In other embodiments, the communication interface 122 may be separately attached to the external surface of the therapy unit 110, and may include a receiver configured to receive data related to the operation of the therapy unit 110.

In some embodiments, the communication interface 122 of the therapy unit 110 may be configured to transmit data through the communications network 104 to the monitoring center 106. Additionally or alternatively, in some embodiments, the data from the therapy unit 110 may be transmitted to the monitoring center 106 by the mobile telecommunications device 112 after receiving, and in some cases processing, the data from the communication interface 122 of the therapy unit 110. The monitoring center 106 may be designed to receive data related to the operation of the therapy system 102, and more specifically, the therapy unit 110 and the negative-pressure source 120. The monitoring center 106 may implement a processing device loaded with software algorithms for processing the data related to the operation and performance of the therapy system 102.

The communication interface 122 of the therapy unit 110 may be configured to communicate with the mobile telecommunications device 112, as well as with the communications network 104 of the therapy network 100. The communication interface 122 may therefore include a transceiver. In one preferred embodiment, the communication interface 122 may include a cellular modem and may be configured to communicate with the communications network 104 through a cellular connection. In some embodiments, the communication interface 122 may include a Bluetooth® radio, ZigBee® radio, or other wireless radio technology for communicating with the mobile telecommunications device 112 and/or the communications network 104 through a personal area network (PAN) or a wide personal area network (WPAN). For example, communications with the communication interface 122 of the therapy unit 110 may be through a synchronized secure interface such as available through a cellular, WIFI, or Bluetooth® connection to the mobile telecommunications device 112. In some embodiments, the mobile telecommunications device 112 may receive data transmitted from the communication interface 122 and may then be utilized to transmit information to and from a remote server, which may allow for remote monitoring of use and performance of the therapy unit 110. The communication interface 122 may be configured to transmit data related to the operation of the therapy system 102, which may occur at a patient's home or at a treatment center, such as a hospital or physician's office. The therapy unit 110 may optionally include a communications port (not shown) for providing wired communication capability, in the event that wireless communications are either unavailable or malfunctioning.

In some embodiments, the communication interface 122 may include a processor that is configured with an algorithm for processing the data related to the operation of the therapy system 102, and more specifically, the negative-pressure source 120 of the therapy unit 110. The processor and associated algorithm(s) may be further capable of transmitting data received from the electronics of the therapy unit 110, including electronics associated with the negative-pressure source 120, to the external communication networks 104. In some embodiments, the communication interface 122 may be further configured to do some stages of processing of the data before transmitting it through the external communications network 104 to either the mobile telecommunications device 112 and/or monitoring center 106.

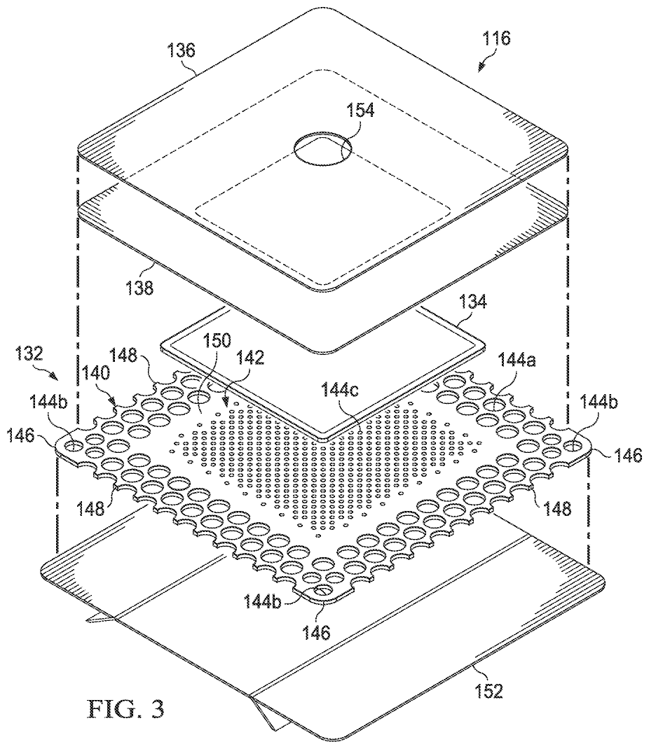

Referring now to FIG. 3, additional details associated with some example embodiments of the cover 116 of FIGS. 1 and 2 are described. For example, the base layer 132 may have a base layer periphery 140 surrounding a central portion 142, and a plurality of apertures 144 disposed throughout the base layer periphery 140 and the central portion 142. The base layer 132 may also have corners 146 and edges 148. The corners 146 and edges 148 may be part of the base layer periphery 140. One of the edges 148 may meet another of the edges 148 to define one of the corners 146. Further, the base layer 132 may have a border 150 substantially surrounding the central portion 142 and positioned between the central portion 142 and the base layer periphery 140. The border 150 may be free of the apertures 144. The base layer 132 may be adapted to cover the tissue interface 114 and the tissue surrounding the tissue site 124, such that the central portion 142 of the base layer 132 is positioned adjacent to or proximate to the tissue interface 114, and the base layer periphery 140 is positioned adjacent to or proximate to tissue surrounding the tissue site 124. In this manner, the base layer periphery 140 may surround the tissue interface 114. Further, the apertures 144 in the base layer 132 may be in fluid communication with the tissue interface 114 and tissue surrounding the tissue site 124.

The apertures 144 in the base layer 132 may have any shape, such as, for example, circles, squares, stars, ovals, polygons, slits, complex curves, rectilinear shapes, triangles, or other shapes. The apertures 144 may be formed by cutting, by application of local RF energy, or other suitable techniques for forming an opening. As shown in FIG. 3, each of the apertures 144 of the plurality of apertures 144 may be substantially circular in shape, having a diameter and an area. The area of each of the apertures 144 may refer to an open space or open area defining each of the apertures 144. The diameter of each of the apertures 144 may define the area of each of the apertures 144. The area of the apertures 144 described in the illustrative embodiments herein may be substantially similar to the area in other embodiments (not shown) for the apertures 144 that may have non-circular shapes. The diameter of each of the apertures 144 may be substantially the same, or each of the diameters may vary depending, for example, on the position of the apertures 144 in the base layer 132. For example, the diameter of the apertures 144 in the base layer periphery 140 may be larger than the diameter of the apertures 144 in the central portion 142 of the base layer 132. Further, the diameter of each of the apertures 144 may be between about 1 millimeter to about 50 millimeters. In some embodiments, the diameter of each of the apertures 144 may be between about 1 millimeter to about 20 millimeters. The apertures 144 may have a uniform pattern or may be randomly distributed on the base layer 132. The size and configuration of the apertures 144 may be designed to control the adherence of the cover 116 to the epidermis 126 as described below.

Still referring primarily to FIG. 3, in some embodiments, the apertures 144 positioned in the base layer periphery 140 may be apertures 144a, the apertures 144 positioned at the corners 146 of the base layer periphery 140 may be apertures 144b, and the apertures 144 positioned in the central portion 142 may be apertures 144c. In some embodiments, the apertures 144a may have a diameter between about 9.8 millimeters to about 10.2 millimeters. The apertures 144b may have a diameter between about 7.75 millimeters to about 8.75 millimeters. The apertures 144c may have a diameter between about 1.8 millimeters to about 2.2 millimeters. The diameter of each of the apertures 144a may be separated from one another by a distance of between about 2.8 millimeters to about 3.2 millimeters. Further, the diameter of at least one of the apertures 144a may be separated from the diameter of at least one of the apertures 144b by approximately a distance of about 2.8 millimeters to about 3.2 millimeters. The diameter of each of the apertures 144b may also be separated from one another by a similar distance. Additionally, a center of one of the apertures 144c may be separated from a center of another of the apertures 144c in a first direction by a distance of between about 2.8 millimeters to about 3.2 millimeters. In a second direction transverse to the first direction, the center of one of the apertures 144c may be separated from the center of another of the apertures 144c by a distance of between about 2.8 millimeters to about 3.2 millimeters. As shown in FIG. 3, the distances may be increased for the apertures 144c in the central portion 142 being positioned proximate to or at the border 150 as compared to the apertures 144c positioned away from the border 150.

As shown in FIG. 3, the central portion 142 of the base layer 132 may be substantially square with each side of the central portion 142 having a length of between about 100 millimeters to about 140 millimeters. In some embodiments, the length may be between about 106 millimeters to about 108 millimeters. The border 150 of the base layer 132 may have a width of between about 4 millimeters to about 11 millimeters and may substantially surround the central portion 142 and the apertures 144c in the central portion 142. In some embodiments, the width may be between about 9 millimeters to about 10 millimeters. The base layer periphery 140 may have a width between about 25 millimeters to about 55 millimeters and may substantially surround the border 150 and the central portion 142. In some embodiments, the width may be between about 26 millimeters to about 28 millimeters. Further, the base layer periphery 140 may have a substantially square exterior with each side of the exterior having a length of between about 120 millimeters to about 200 millimeters. In some embodiments, the length may be between about 176 millimeters and about 184 millimeters. Although FIG. 3 depicts the central portion 142, the border 150, and the base layer periphery 140 of the base layer 132 as having a substantially square shape, these and other components of the base layer 132 may have any shape to suit a particular application. Further, the dimensions of the base layer 132 as described herein may be increased or decreased, for example, substantially in proportion to one another to suit a particular application. The use of the dimensions in the proportions described above may enhance the cosmetic appearance of a tissue site. For example, these proportions may provide a surface area for the base layer 132, regardless of shape, that is sufficiently smooth to enhance the movement and proliferation of epithelial cells at the tissue site 124, and reduce the likelihood of granulation tissue in-growth into the dressing 108.

The base layer 132 may be a soft, pliable material suitable for providing a fluid seal with the tissue site 124 as described herein. For example, the base layer 132 may comprise a silicone gel, a soft silicone, hydrocolloid, hydrogel, polyurethane gel, polyolefin gel, hydrogenated styrenic copolymer gel, a foamed gel, a soft closed-cell foam such as polyurethanes and polyolefins coated with an adhesive as described below, polyurethane, polyolefin, or hydrogenated styrenic copolymers. The base layer 132 may have a thickness between about 500 microns (mm) and about 1000 microns (mm). In some embodiments, the base layer 132 may have a stiffness between about 5 Shore OO and about 80 Shore OO. Further, in some embodiments, the base layer 132 may be comprised of hydrophobic or hydrophilic materials.

In some embodiments (not shown), the base layer 132 may be a hydrophobic-coated material. For example, the base layer 132 may be formed by coating a spaced material, such as, for example, woven, nonwoven, molded, or extruded mesh with a hydrophobic material. The hydrophobic material for the coating may be a soft silicone, for example. In this manner, the adhesive 138 may extend through openings in the spaced material analogous to the apertures 144.

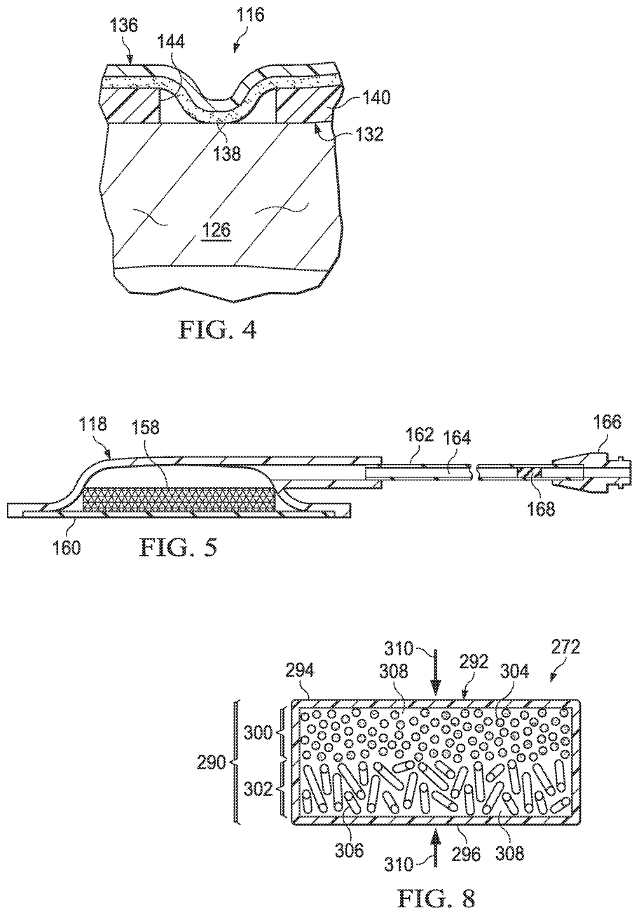

The adhesive 138 may be in fluid communication with the apertures 144 in at least the base layer periphery 140. In this manner, the adhesive 138 may be in fluid communication with tissue surrounding the tissue site 124 through the apertures 144 in the base layer 132. As described in further detail below and shown in FIG. 4, the adhesive 138 may extend or be passed through the plurality of apertures 144 to contact the epidermis 126 for securing the cover 116 to, for example, the tissue surrounding the tissue site 124. The apertures 144 may provide sufficient contact of the adhesive 138 to the epidermis 126 to secure the cover 116 about the tissue site 124. However, the configuration of the apertures 144 and the adhesive 138, described below, may permit release and repositioning of the cover 116 about the tissue site 124.

In some embodiments, an additional or alternative attachment device may be used to secure the cover 116 about the tissue site 124. For example, double-sided tape, paste, hydrocolloid, hydrogel, silicone gel, or organogel may be used. Furthermore, thicker adhesives, or combinations of adhesives, may be applied in some embodiments to improve seals and to reduce leaks.

At least one of the apertures 144a in the base layer periphery 140 may be positioned at the edges 148 of the base layer periphery 140, and may have an interior cut open or exposed at the edges 148 that is in fluid communication in a lateral direction with the edges 148. The lateral direction may refer to a direction toward the edges 148 and in the same plane as the base layer 132. As shown in FIG. 3, a plurality of the apertures 144a in the base layer periphery 140 may be positioned proximate to or at the edges 148 and in fluid communication in a lateral direction with the edges 148. The apertures 144a positioned proximate to or at the edges 148 may be spaced substantially equidistant around the base layer periphery 140 as shown in FIG. 3. However, in some embodiments, the spacing of the apertures 144a proximate to or at the edges 148 may be irregular. The adhesive 138 may be in fluid communication with the edges 148 through the apertures 144a being exposed at the edges 148. In this manner, the apertures 144a at the edges 148 may permit the adhesive 138 to flow around the edges 148 for enhancing the adhesion of the edges 148 around the tissue site 124, for example.

Continuing with FIG. 3, the apertures 144b at the corners 146 of the base layer periphery 140 may be smaller than the apertures 144a in other portions of the base layer periphery 140. For a given geometry of the corners 146, the smaller size of the apertures 144b compared to the apertures 144a may maximize the surface area of the adhesive 138 exposed and in fluid communication through the apertures 144b at the corners 146. For example, the edges 148 may intersect at substantially a right angle, or about 90 degrees, to define the corners 146. Also as shown, the corners 146 may have a radius of about 10 millimeters. Three of the apertures 144b having a diameter between about 7.75 millimeters to about 8.75 millimeters may be positioned in a triangular configuration at the corners 146 to maximize the exposed surface area for the adhesive 138. The size and number of the apertures 144b in the corners 146 may be adjusted as necessary, depending on the chosen geometry of the corners 146, to maximize the exposed surface area of the adhesive 138 as described above. Further, the apertures 144b at the corners 146 may be fully housed within the base layer 132, substantially precluding fluid communication in a lateral direction exterior to the corners 146. The apertures 144b at the corners 146 being fully housed within the base layer 132 may substantially preclude fluid communication of the adhesive 138 exterior to the corners 146, and may provide improved handling of the cover 116 during deployment at the tissue site 124. Further, the exterior of the corners 146 being substantially free of the adhesive 138 may increase the flexibility of the corners 146 to enhance comfort.

Similar to the apertures 144b in the corners 146, any of the apertures 144 may be adjusted in size and number to maximize the surface area of the adhesive 138 in fluid communication through the apertures 144 for a particular application or geometry of the base layer 132. For example, in some embodiments (not shown), the apertures 144b, or apertures of another size, may be positioned in the base layer periphery 140 and at the border 150. Similarly, the apertures 144b, or apertures of another size, may be positioned as described above in other locations of the base layer 132 that may have a complex geometry or shape.

The adhesive 138 may be a medically-acceptable adhesive. The adhesive 138 may also be flowable. For example, the adhesive 138 may comprise an acrylic adhesive, rubber adhesive, high-tack silicone adhesive, polyurethane, or other adhesive substance. In some embodiments, the adhesive 138 may be a pressure-sensitive adhesive comprising an acrylic adhesive with coating weight of 15 grams/m² (gsm) to 70 grams/m² (gsm). The adhesive 138 may be a layer having substantially the same shape as the base layer periphery 140 as shown in FIG. 3. In some embodiments, the layer of the adhesive 138 may be continuous or discontinuous. Discontinuities in the adhesive 138 may be provided by apertures (not shown) in the adhesive 138. Apertures in the adhesive 138 may be formed after application of the adhesive 138 or by coating the adhesive 138 in patterns on a carrier layer, such as, for example, a side of the sealing member 136 adapted to face the epidermis 126. Further, apertures in the adhesive 138 may be sized to control the amount of the adhesive 138 extending through the apertures 144 in the base layer 132 to reach the epidermis 126. Apertures in the adhesive 138 may also be sized to enhance the Moisture Vapor Transfer Rate (MVTR) of the cover 116, described in further detail below.

Factors that may be utilized to control the adhesion strength of the cover 116 may include the diameter and number of the apertures 144 in the base layer 132, the thickness of the base layer 132, the thickness and amount of the adhesive 138, and the tackiness of the adhesive 138. An increase in the amount of the adhesive 138 extending through the apertures 144 may correspond to an increase in the adhesion strength of the cover 116. A decrease in the thickness of the base layer 132 may correspond to an increase in the amount of adhesive 138 extending through the apertures 144. Thus, the diameter and configuration of the apertures 144, the thickness of the base layer 132, and the amount and tackiness of the adhesive utilized may be varied to provide a desired adhesion strength for the cover 116. For example, in some embodiments, the thickness of the base layer 132 may be about 200 microns, the layer of adhesive 138 may have a thickness of about 30 microns and a tackiness of 2000 grams per 25 centimeter wide strip, and the diameter of the apertures 144a in the base layer 132 may be about 10 millimeters.

In some embodiments, the tackiness of the adhesive 138 may vary in different locations of the base layer 132. For example, in locations of the base layer 132 where the apertures 144 are comparatively large, such as the apertures 144a, the adhesive 138 may have a lower tackiness than other locations of the base layer 132 where the apertures 144 are smaller, such as the apertures 144b and 144c. In this manner, locations of the base layer 132 having larger apertures 144 and lower tackiness adhesive 138 may have an adhesion strength comparable to locations having smaller apertures 144 and adhesive 138 having a higher tackiness.

Clinical studies have shown that the configuration described herein for the base layer 132 and the adhesive 138 may reduce the occurrence of blistering, erythema, and leakage when in use. Such a configuration may provide, for example, increased patient comfort and increased durability of the cover 116, as well as overall dressing 108.

Still referring to the embodiment of FIG. 3, a release liner 152 may be attached to or positioned adjacent to the base layer 132 to protect the adhesive 138 prior to application of the cover 116 to the tissue site 124. Prior to application of the cover 116 to the tissue site 124, the base layer 132 may be positioned between the sealing member 136 and the release liner 152. Removal of the release liner 152 may expose the base layer 132 and the adhesive 138 for application of the cover 116 to the tissue site 124. The release liner 152 may also provide stiffness to assist with, for example, deployment of the cover 116. The release liner 152 may be, for example, a casting paper, a film, or polyethylene. Further, the release liner 152 may be a polyester material such as polyethylene terephthalate (PET), or similar polar semi-crystalline polymer. The use of a polar semi-crystalline polymer for the release liner 152 may substantially preclude wrinkling or other deformation of the cover 116. For example, the polar semi-crystalline polymer may be highly orientated and resistant to softening, swelling, or other deformation that may occur when brought into contact with components of the cover 116, or when subjected to temperature or environmental variations, or sterilization. Further, a release agent may be disposed on a side of the release liner 152 that is configured to contact the base layer 132. For example, the release agent may be a silicone coating and may have a release factor suitable to facilitate removal of the release liner 152 by hand and without damaging or deforming the cover 116. In some embodiments, the release agent may be fluorosilicone. In other embodiments, the release liner 152 may be uncoated or otherwise used without a release agent.

Continuing with FIGS. 2 and 3, the sealing member 136 may have a sealing member periphery 137 and a sealing member central portion 139. The sealing member 136 may additionally include a sealing member aperture 154, as described below. The sealing member periphery 137 may be positioned proximate to base layer periphery 140 such that the sealing member central portion 139 and the central portion 142 of the base layer 132 define an enclosure. The adhesive 138 may be positioned at least between the sealing member periphery 137 and the base layer periphery 140. The sealing member 136 may cover the tissue site 124 and the tissue interface 114 to provide a fluid seal and a sealed space between the tissue site 124 and the sealing member 136. Further, the sealing member 136 may cover other tissue, such as a portion of the epidermis 126, surrounding the tissue site 124 to provide the fluid seal between the sealing member 136 and the tissue site 124. In some embodiments, a portion of the sealing member periphery 137 may extend beyond the base layer periphery 140 and into direct contact with tissue surrounding the tissue site 124. In some embodiments, the sealing member periphery 137, for example, may be positioned in contact with tissue surrounding the tissue site 124 to provide the sealed space without the base layer 132. Thus, the adhesive 138 may also be positioned at least between the sealing member periphery 137 and tissue, such as the epidermis 126, surrounding the tissue site 124. The adhesive 138 may be disposed on a surface of the sealing member 136 adapted to face the tissue site 124 and the base layer 132.

The sealing member 136 may be formed from any material that allows for a fluid seal. A fluid seal may be a seal adequate to maintain reduced pressure at a desired site given the particular reduced pressure source or system involved. The sealing member 136 may comprise, for example, one or more of the following materials: hydrophilic polyurethane; cellulosics; hydrophilic polyamides; polyvinyl alcohol; polyvinyl pyrrolidone; hydrophilic acrylics; hydrophilic silicone elastomers; an INSPIRE 2301 material from Expopack Advanced Coatings of Wrexham, United Kingdom having, for example, an MVTR (inverted cup technique) of 14400 g/m²/₂/24 hours and a thickness of about 30 microns; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; polyurethane (PU); EVA film; co-polyester; silicones; a silicone drape; a 3M Tegaderm® drape; a polyurethane (PU) drape such as one available from Avery Dennison Corporation of Pasadena, Calif.; polyether block polyamide copolymer (PEBAX), for example, from Arkema, France; Expopack 2327; or other appropriate material.

The sealing member 136 may be vapor permeable and liquid impermeable, thereby allowing vapor and inhibiting liquids from exiting the sealed space provided by the dressing 108. In some embodiments, the sealing member 136 may be a flexible, breathable film, membrane, or sheet having a high MVTR of, for example, at least about 300 g/m² per 24 hours. In other embodiments, a low or no vapor transfer drape might be used. The sealing member 136 may comprise a range of medically suitable films having a thickness between about 15 microns (μm) to about 50 microns (μm).

The absorbent member 134 may be a hydrophilic material adapted to absorb fluid from, for example, the tissue site 124. Materials suitable for the absorbent layer 134 may include, without limitation, Luquafleece® material, Texsus FP2326, BASF 402C, Technical Absorbents 2317 available from Technical Absorbents (www.techabsorbents.com), sodium polyacrylate super absorbers, cellulosics (carboxy methyl cellulose and salts such as sodium CMC), or alginates. In some embodiments, the absorbent member 134 may be a plurality of absorbent layers. The absorbent member 134 may include, without limitation, any number of individual absorbent components as desired for treating a particular tissue site. Including additional absorbent components as part of the absorbent member 134 may increase the absorbent mass of the cover 116 and generally provide greater fluid capacity. However, for a given absorbent mass, multiple light coat-weight absorbent layers may be used rather than a single heavy coat-weight absorbent layer to provide a greater absorbent surface area for further enhancing the absorbent efficiency of the absorbent member 134.

Referring now also to FIG. 4, the figures illustrate how the cover 116 may be applied over the tissue interface 114 and the tissue site 124 to form a sealed space. Specifically, the base layer 132 may be applied covering the tissue interface 114 and tissue surrounding the tissue site 124. The materials described above for the base layer 132 may have a tackiness that may hold the tissue interface 114 initially in position. The tackiness may be such that if an adjustment is desired, the cover 116 may be removed and reapplied. Once the components of the dressing 108 are in the desired position, a force may be applied, such as hand pressure, on a side of the sealing member 136 opposite the tissue site 124. The force applied to the sealing member 136 may cause at least some portion of the adhesive 138 to penetrate or extend through the plurality of apertures 144 and into contact with tissue surrounding the tissue site 124, such as the epidermis 126, to releasably adhere the cover 116 about the tissue site 124. In this manner, the configuration of the cover 116 described above may provide an effective and reliable seal against challenging anatomical surfaces, such as an elbow or heel, at and around the tissue site 124. Further, the cover 116 may permit re-application or re-positioning to, for example, correct air leaks caused by creases and other discontinuities in the cover 116 and the tissue site 124. The ability to rectify leaks may increase the reliability of the therapy and reduce power consumption of the therapy unit 110.

Referring now to FIG. 5, the conduit interface 118 may be positioned proximate to the sealing member 136, as shown in FIG. 2. The conduit interface 118 may be in fluid communication with the cover 116 through the sealing member aperture 154 to provide negative pressure from the negative-pressure source 120 to the dressing 108. In some embodiments, the conduit interface 118 may also be adapted to be positioned in fluid communication with the tissue interface 114.

The conduit interface 118 may comprise a medical-grade, soft polymer or other pliable material. As non-limiting examples, the conduit interface 118 may be formed from polyurethane, polyethylene, polyvinyl chloride (PVC), fluorosilicone, or ethylene-propylene. In some illustrative, non-limiting embodiments, conduit interface 118 may be molded from DEHP-free PVC. The conduit interface 118 may be formed in any suitable manner such as by molding, casting, machining, or extruding. Further, the conduit interface 118 may be formed as an integral unit or as individual components and may be coupled to the cover 116 by, for example, adhesive or welding.

In some embodiments, the conduit interface 118 may be formed of an absorbent material having absorbent and evaporative properties. The absorbent material may be vapor permeable and liquid impermeable, thereby being configured to permit vapor to be absorbed into and evaporated from the material through permeation while inhibiting permeation of liquids. The absorbent material may be, for example, a hydrophilic polymer such as a hydrophilic polyurethane. Although the term hydrophilic polymer may be used in the illustrative embodiments that follow, any absorbent material having the properties described herein may be suitable for use in the therapy system 102. Further, the absorbent material or hydrophilic polymer may be suitable for use in various components of the therapy system 102 as described herein.

The use of such a hydrophilic polymer for the conduit interface 118 may permit liquids in the conduit interface 118 to evaporate, or otherwise dissipate, during operation. For example, the hydrophilic polymer may allow the liquid to permeate or pass through the conduit interface 118 as vapor, in a gaseous phase, and evaporate into the atmosphere external to the conduit interface 118. Such liquids may be, for example, condensate or other liquids. Condensate may form, for example, as a result of a decrease in temperature within the conduit interface 118, or other components of the therapy system 102, relative to the temperature at the tissue site 124. Removal or dissipation of liquids from the conduit interface 118 may increase visual appeal and prevent odor. Further, such removal of liquids may also increase efficiency and reliability by reducing blockages and other interference with the components of the therapy system 102.

In some embodiments, the absorbent material or hydrophilic polymer may have an absorbent capacity in a saturated state that is substantially equivalent to the mass of the hydrophilic polymer in an unsaturated state. The hydrophilic polymer may be fully saturated with vapor in the saturated state and substantially free of vapor in the unsaturated state. In both the saturated state and the unsaturated state, the hydrophilic polymer may retain substantially the same physical, mechanical, and structural properties. For example, the hydrophilic polymer may have a hardness in the unsaturated state that is substantially the same as a hardness of the hydrophilic polymer in the saturated state. The hydrophilic polymer and the components of the therapy system 102 incorporating the hydrophilic polymer may also have a size that is substantially the same in both the unsaturated state and the saturated state. Further, the hydrophilic polymer may remain dry, cool to the touch, and pneumatically sealed in the saturated state and the unsaturated state. The hydrophilic polymer may also remain substantially the same color in the saturated state and the unsaturated state. In this manner, this hydrophilic polymer may retain sufficient strength and other physical properties to remain suitable for use in the therapy system 102. An example of such a hydrophilic polymer is offered under the trade name Techophilic HP-93A-100, available from The Lubrizol Corporation of Wickliffe, Ohio, United States. Techophilic HP-93A-100 is an absorbent hydrophilic thermoplastic polyurethane capable of absorbing 100% of the unsaturated mass of the polyurethane in water and having a durometer or Shore Hardness of about 83 Shore A.

In some embodiments, the conduit interface 118 may carry an odor filter 158 adapted to substantially preclude the passage of odors from the tissue site 124. Further, the conduit interface 118 may carry a primary hydrophobic filter 160 adapted to substantially preclude the passage of liquids from the cover 116 and tissue site 124. The odor filter 158 and the primary hydrophobic filter 160 may be disposed in the conduit interface 118 or other suitable location such that fluid communication between the negative-pressure source 120 and the dressing 108 is provided through the odor filter 158 and the primary hydrophobic filter 160. In some embodiments, the odor filter 158 and the primary hydrophobic filter 160 may be secured within the conduit interface 118 in any suitable manner, such as by adhesive or welding. In some embodiments, the odor filter 158 and the primary hydrophobic filter 160 may be positioned in any exit location in the dressing 108 that is in fluid communication with the atmosphere or the negative-pressure source 120. The odor filter 158 may also be positioned in any suitable location in the therapy system 102 that is in fluid communication with the tissue site 124.

The odor filter 158 may be comprised of a carbon material in the form of a layer or particulate. For example, the odor filter 158 may comprise a woven carbon cloth filter such as those manufactured by Chemviron Carbon, Ltd. of Lancashire, United Kingdom (www.chemvironcarbon.com). The primary hydrophobic filter 160 may be comprised of a material that is liquid impermeable and vapor permeable. For example, the primary hydrophobic filter 160 may comprise a material manufactured under the designation MMT-314 by W.L. Gore & Associates, Inc. of Newark, Del., United States, or similar materials. The primary hydrophobic filter 160 may be provided in the form of a membrane or layer.

Still referring to FIG. 5, a conduit 162 having an internal lumen 164 may be coupled in fluid communication between the negative-pressure source 120 and the dressing 108. The internal lumen 164 may have an internal diameter between about 0.5 millimeters to about 3.0 millimeters. In some embodiments, the internal diameter of the internal lumen 164 may be between about 1 millimeter to about 2 millimeters. The conduit interface 118 may be coupled in fluid communication with the cover 116 and adapted to connect between the conduit 162 and the cover 116 of the dressing 108 for providing fluid communication with the negative-pressure source 120. The conduit interface 118 may be fluidly coupled to the conduit 162 in any suitable manner, such as, for example, by an adhesive, solvent or non-solvent bonding, welding, or interference fit. The sealing member aperture 154 may provide fluid communication between the cover 116 and the conduit interface 118. In some embodiments, the conduit 162 may be inserted into the dressing 108 through the sealing member aperture 154 to provide fluid communication with the negative-pressure source 120 without use of the conduit interface 118. The negative-pressure source 120 may also be directly coupled in fluid communication with the dressing 108 or the sealing member 136 without use of the conduit 162. In some embodiments, the conduit 162 may be, for example, a flexible, extruded polymer tube. A distal end of the conduit 162 may include a coupling 166 for attachment to the negative-pressure source 120.

The conduit 162 may have a secondary hydrophobic filter 168 disposed in the internal lumen 164, such that fluid communication between the negative-pressure source 120 and the dressing 108 is provided through the secondary hydrophobic filter 168. The secondary hydrophobic filter 168 may be, for example, a porous, sintered polymer cylinder sized to fit the dimensions of the internal lumen 164 to substantially preclude liquid from bypassing the cylinder. The secondary hydrophobic filter 168 may also be treated with an absorbent material adapted to swell when brought into contact with liquid to block the flow of the liquid. The secondary hydrophobic filter 168 may be positioned at any location within the internal lumen 164. However, positioning the secondary hydrophobic filter 168 within the internal lumen 164 closer toward the negative-pressure source 120, rather than the dressing 108, may allow a user to detect the presence of liquid in the internal lumen 164.

In some embodiments, the conduit 162 and the coupling 166 may be formed of an absorbent material or a hydrophilic polymer as described above for the conduit interface 118. In this manner, the conduit 162 and the coupling 166 may permit liquids in the conduit 162 and the coupling 166 to evaporate, or otherwise dissipate, as described above for the conduit interface 118. The conduit 162 and the coupling 166 may be, for example, molded from the hydrophilic polymer separately, as individual components, or together as an integral component. Further, a wall of the conduit 162 defining the internal lumen 164 may be extruded from the hydrophilic polymer. The conduit 162 may be less than about 1 meter in length, but may have any length to suit a particular application. In some embodiments, a length of about 1 foot or 304.8 millimeters may provide enough absorbent and evaporative surface area to suit many applications, and may provide a cost savings compared to longer lengths. If an application requires additional length for the conduit 162, the absorbent hydrophilic polymer may be coupled in fluid communication with a length of conduit formed of a non-absorbent hydrophobic polymer to provide additional cost savings.

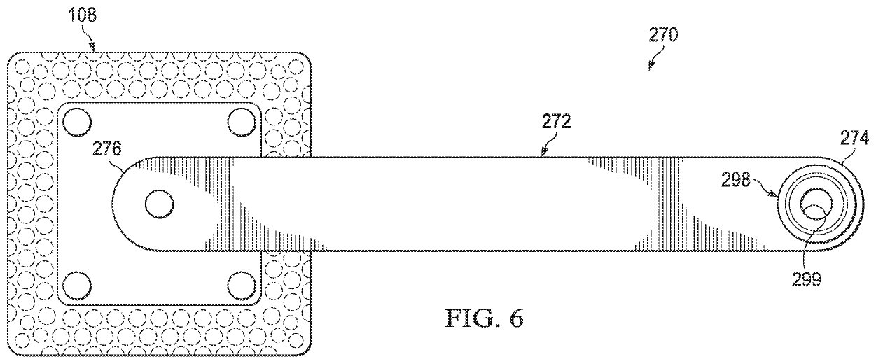

Referring to FIG. 6, in some embodiments, a low-profile conduit assembly 270 may be coupled in fluid communication between the dressing 108 and the negative-pressure source 120 in any suitable manner. In some embodiments, the low-profile conduit assembly 270 may include a low-profile conduit 272 having a receiving end 274 and a transmitting end 276 separated by a fluid conductor. The length of the low-profile conduit 272 may be between about 300 millimeters to about 1200 millimeters, or any other length suitable for a particular application.

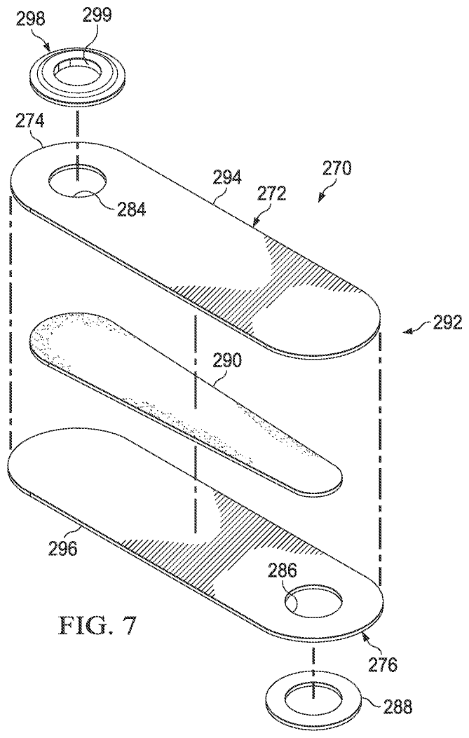

Referring now also to FIG. 7, the receiving end 274 of the low-profile conduit 272 may have a receiving end aperture 284, and the transmitting end 276 may have a transmitting end aperture 286. The receiving end 274 and the receiving end aperture 284 may be in fluid communication with the transmitting end 276 and the transmitting end aperture 286 through the length of the low-profile conduit 272. A seal 288 may be positioned about the transmitting end aperture 286 and between the transmitting end 276 and the dressing 108 for bonding the transmitting end 276 to the dressing 108 and in fluid communication with the dressing 108 through the transmitting end aperture 286.

The low-profile conduit 272 may additionally include a manifold material 290 encapsulated or sealingly enclosed within a conduit sealing member 292. The manifold material 290 may be encapsulated or sealingly enclosed with the conduit sealing member 292 between the receiving end 274 and the transmitting end 276 of the low-profile conduit 272. For example, in some embodiments, the conduit sealing member 292 may include a first sealing layer 294 and a second sealing layer 296. The first sealing layer 294 may have a first periphery bonded to a second periphery of the second sealing layer 296 around the manifold material 290 in any suitable manner for forming the conduit sealing member 292 and encapsulating the manifold material 290 therein. The conduit sealing member 292 may be comprised of similar materials described above for the sealing member 136. For example, the conduit sealing member 292 may be an adhesive coated film, such as an Inspire 2327 drape. As should be apparent from the above description, the structure of the low-profile conduit 272 may, in some embodiments, eliminate the need for including more traditional conduit structures and materials, such as plastic tube sets.

In some embodiments, the second sealing layer 296 may include an adhesive layer on an external, tissue-facing surface. For example, the external surface of the sealing layer 296 may further include a layer of a double-sided adhesive tape, which may have a release liner protecting the external adhesive surface prior to application to the skin of a patient. In some instances, the release liner may be segmented, so that only selected portions may be removed to expose the underlying adhesive, as desired, while some embodiments may include adhesive layers that provide an adhesive tack only in specific areas. The adhesive layer may include any adhesive material, including adhesive acrylates, however those adhesives that provide an adequate tack without causing harm to a patient's skin may be most appropriate.

The example embodiments of FIGS. 6 and 7 are shown with an attachment port 298 for physically attaching and fluidly connecting a source of negative pressure, such as the therapy unit 110, to the low-profile conduit assembly 270. For example, in some embodiments, the attachment port 298 may include snap assembly 299 for attaching to a compatible snap assembly located on a surface of a therapy unit, such as therapy unit 110. Alternatively or additionally, the attachment port 298 may include other forms of attachment mechanisms, such as hook and loop devices, adhesives, straps, elastic bands, or other means for attaching a therapy unit to the receiving end 274 of the low-profile conduit assembly 270. In some alternative embodiments, rather than including the attachment port 298, the receiving end 274 of the low-profile conduit assembly 270 may be coupled to a conduit, which may terminate in an adapter for fluid connection to the negative-pressure source 120.

Referring now to FIG. 8, the manifold material 290 may include a distribution layer 300 and an acquisition layer 302. The distribution layer 300 may be comprised of longitudinal fibers 304. The longitudinal fibers 304 may be oriented substantially in a longitudinal direction along the length of the low-profile conduit 272. The acquisition layer 302 may be comprised of vertical fibers 306. The vertical fibers 306 may be oriented substantially vertical or normal relative to the longitudinal fibers 304 and the length of the low-profile conduit 272. The distribution layer 300 may be coupled to the acquisition layer 302. Fluid communication voids 308 may be located or defined between and among the longitudinal fibers 304 of the distribution layer 300 and the vertical fibers 306 of the acquisition layer 302. The fluid communication voids 308 may provide fluid communication through the manifold material 290 and the low-profile conduit 272 even when exposed to a force, such as a compression force depicted in FIG. 8 as arrows 310, for example. When exposed to such a force, the longitudinal fibers 304 and the vertical fibers 306 may engage one another to substantially preclude blockage, closure, or other interference with the fluid communication voids 308 in providing fluid communication through the low-profile conduit 272.

The manifold material 290 may be a non-woven material such as, for example, a polyester non-woven or Libeltex TDL4. In some embodiments, other non-woven structures may be used for the manifold material 290, such as Libeltex TDL2, or laminations with fiber or foam structures. Further, other materials for the conduit sealing member 292 may be used, such as polyurethane film, films with and without adhesive, and high Moisture Vapor Transfer Rate (MVTR) films. The high MVTR films may provide for evaporation of condensate. In some preferred embodiments, the low-profile conduit 272 may be a substantially flat structure made from a thin, flexible non-woven manifold material 290 sealed between two layers of conduit sealing member 292, which may be polyurethane layers or other occlusive layers which may be bonded or sealed together.

In some embodiments, patterns or shallow ridges may be embossed into the conduit sealing member 292 to aid pressure transfer and further resist crushing. Further, odor adsorbing additives may be added to the low-profile conduit 272 to absorb bad smelling gases and vapors that may be liberated form the wound or dressing.

The low-profile conduit 272 may offer considerable advantages in operation. For example, the low-profile conduit 272 may provide a flexible pressure transfer conduit capable of transmitting negative pressure when exposed to a force, such as a crushing force or compression force. The low-profile conduit 272 may be exposed to such forces, for example, from a patient sitting, rolling, or standing on the low-profile conduit 272. The low-profile conduit 272 may also experience a force or compression force from being kinked or folded. However, the configuration of the low-profile conduit 272 may also provide for the transmission of negative pressure when kinked or folded. Further, the low-profile conduit 272 may enable the caregiver to choose a route from the dressing 108, or another dressing, to the negative-pressure source 120 that is best or convenient for the patient, rather than being limited to routes less susceptible to being crushed and better suited for transfer of pressure. The low-profile conduit 272 may present less risk of causing discomfort or pressure-point related injuries to the patient. The flexibility and conformability of the low-profile conduit 272 may enable it to be folded, permitting smaller packaging pouches to be used. Additionally, the low-profile conduit 272 can be disguised or camouflaged to blend in with the patient's clothing or attire. Further, the low-profile conduit 272 may be folded, if too long, and located on the patient's skin by tape or other commonly-used skin adhesives, in order to prevent the excess conduit material from becoming a trip hazard.

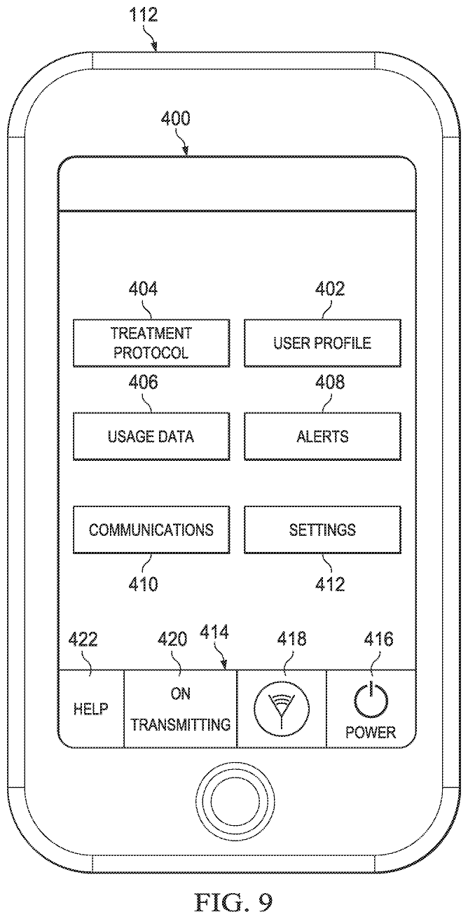

FIG. 9 illustrates an example embodiment of a display of the mobile telecommunications device 112. The mobile telecommunications device 112 may be configured to display a graphical user interface (GUI) 400. The GUI 400 may provide a user with an interface to a software application operable on the mobile telecommunications device 112. The GUI 400 may include a number of selectable graphical elements, including a “user profile” soft-button 402, “treatment protocol” soft-button 404, “usage data” soft-button 406, “alerts” soft-button 408, “communications” soft-button 410, and “settings” soft-button 412, along with soft-buttons assigned to any other features related to the treatment of a tissue site. A user may select any of these functions (i.e., user profile, treatment protocol, usage data, alerts, communications, settings) to cause the mobile telecommunications device 112 to present the user with another GUI for performing the selected function. In addition, an “exit” soft-button (not shown) may be available to the user to exit the currently-presented GUI. It should be understood that the GUI 400 is exemplary and that other and/or alternative functions and selection elements may be provided to the user. For example, in some embodiments, the software application associated with the GUI 400 may be integrated with or linked to additional software packages that may provide a range of other functions related to care of a wound site.

An information region 414 of the GUI 400 may include selectable graphical elements and display other information in which the user may be interested. For example, a “power” soft-button 416 may enable a user to selectively turn the negative-pressure source 120 on and off. A “transmit” soft-button 418 may enable a user to selectively activate or deactivate the wireless communication between the therapy unit 110 and the mobile telecommunications device 112 and/or the monitoring center 106. A “status indicator” 420 may provide an indication of whether the mobile telecommunications device 112 is currently exchanging data with the therapy unit 110. The status indicator 420 may also notify the user of current status of the therapy unit 110, including the negative-pressure source 120, as well as the overall therapy system 102. For example, the status indicator 420 may indicate that the negative-pressure source 120 is (i) currently on, (ii) operating according to a specifically-selected treatment protocol, and (iii) transmitting operational data to the mobile telecommunications device 112. The status indicator 420 may also indicate whether the mobile telecommunications device 112 is transmitting through the communications network 104 to the external monitoring center 106. In some embodiments, the status indicator 420 may also indicate whether the negative-pressure source 120 is directly transmitting data through the communications network 104 to an external monitoring center 106. Additionally, a “help” soft-button 422 may be displayed to enable the user to receive help about the negative-pressure source 120, therapy unit 110, or the overall therapy system 102, in addition to the particular functions currently being displayed on the GUI 400.

The information region 414 may also be configured to display messages communicated from a remote party to the user. For example, a clinician at the monitoring center 106 may send messages through the communications network 104 to the mobile telecommunications device 112 for display to a user. Such messages may include instructions for how to keep the therapy system 102 properly operating. Additionally, such messages may include instructions for troubleshooting one or more issues presented with the therapy system 102. Example troubleshooting instructions may include directions to replace a dressing 108, clear a fluid blockage, or recharge or replace a battery of the therapy unit 110.

Components of the systems described herein, such as therapy system 102, may be provided to a user, such as a clinician or patient, in a kit. For example, in some embodiments a kit may include a wound dressing, a low-profile conduit material, such as that used for the low-profile conduit 272, supplied in a roll, two therapy units, each containing a negative-pressure source, and an adhesive material. The wound dressing may be a wound-specific dressing, and thus in some cases, the type of wound dressing included in the kit may vary based on the particular treatment application. The low-profile conduit material may be supplied on a roll containing a length of material, which has not been sized or cut to the requirements of the specific treatment application. Two therapy units can be provided to enable a user to easily swap one out for the other, in order to provide continued therapy while the batteries of the first therapy unit are either being recharged or replaced, for example. Thus, the patient may benefit from continuous therapy and ideally high-levels of compliance with prescribed therapy protocols, which may require a specific number of hours of therapy per day.

During application of the therapy system provided in a kit, the clinician or patient may determine an approximate length of low-profile conduit material required to connect the wound dressing and therapy unit, based on where each will be placed on the patient. The appropriate amount of low-profile conduit material may then be cut from the roll of low-profile conduit material. In some instances, a first end of the cut low-profile conduit material can be positioned on the patient, and the wound dressing can be applied over this first end to secure the low-profile conduit material in fluid communication with the wound dressing. In other instances, the first end of the cut portion of the low-profile conduit material may be fluidly connected to an aperture in the dressing, for example, sealing member aperture 154 of the cover 116, such as by using a seal, such as seal 288. In some instances, the user may wish to apply the adhesive material, which may be provided in the kit in a spray bottle or as a gel, to the areas surrounding the wound site and along the area(s) of the body where the wound dressing and the low-profile conduit material will be applied. A second end of the low-profile conduit material can be positioned at the anatomical location desired for attachment of one of the therapy units provided in the kit. The therapy unit can then be attached to an attachment port of the second end of the low-profile conduit material. The attachment port may attach the therapy unit to the low-profile conduit material. Additionally, if desired or necessary, the therapy unit may be secured to the patient using further attachment means, such as elastic straps.

During operation of the therapy system 102, the negative-pressure source 120 may be activated to provide negative pressure to the dressing 108. For example, in some of the embodiments employing the low-profile conduit assembly 270, negative pressure may be provided to the receiving end 274 of the low-profile conduit 272. The negative pressure may be transmitted through the communication voids 308 in the manifold material 290 to the transmitting end 276, and thus to the dressing 108. Fluid flow associated with the application of the negative pressure may be gaseous and substantially free of liquid. Thus, the low-profile conduit 272 may be substantially free of liquid during operation. Further, the flow rate may be equal or less than about 100 cubic centimeters per minute. In some embodiments, the flow rate may be between about 1 cubic centimeter per minute to about 3 cubic centimeters per minute.

As negative pressure is administered to the dressing 108, fluids from the tissue site 124 may be drawn out into the components of the dressing 108. As the dressing 108 comes into contact with fluid from the tissue site 124, the fluid may move through the tissue interface 114 and in contact with the base layer 132 of the cover 116. The fluid may then pass through some if the apertures 144 of the base layer 132 toward the absorbent member 134. The absorbent member 134 may wick or otherwise move the fluid through the tissue interface 114 and away from the tissue site 124. As described above, the tissue interface 114 may be adapted to communicate fluid from the tissue site 124 rather than store the fluid. Thus, the absorbent member 134 may be more absorbent than the tissue interface 114. The absorbent member 134 being more absorbent than the tissue interface 114 may provide an absorbent gradient through the dressing 108 that attracts fluid from the tissue site 124 or the tissue interface 114 to the absorbent member 134. Thus, in some embodiments, the absorbent member 134 may be adapted to wick, pull, draw, or otherwise attract fluid from the tissue site 124 through the tissue interface 114 to be stored within the absorbent member 134.

The systems, apparatuses, and methods described herein may provide significant advantages. For example, as disclosed herein, the various embodiments of the therapy system 102 may offer a lighter, more portable, and overall more user-friendly negative-pressure wound therapy system. As already discussed in some detail, the therapy system 102, as well as therapy network 100, include several components that may allow increased freedom and mobility. For example, by including a dressing 108 which includes absorbent capabilities, the need for including additional means for exudate storage in the therapy system 102, such as solid containers or pouches, may be eliminated or at least largely reduced. Additionally, as described above, incorporating a mobile-friendly conduit, such as low-profile conduit 272, may offer portability benefits. The use of a simplified therapy unit, such as therapy unit 110, may also allow the size and weight of the portions of the therapy system 102 worn by a patient to be reduced.

The therapy unit 110 may be substantially simplified, and therefore made to be lighter and less cumbersome than many commercially-available units. Several design considerations may be incorporated to achieve this increased portability. For example, removal of components from the therapy unit 110, such as a user interface screen and associated materials and electronics normally required for input/output and control functions, may offer significant weight reduction, in addition to possible cost reduction for the therapy unit 110. Although in some embodiments, the therapy unit 110 may require a communication module for allowing a user to control the device from the mobile telecommunications device 112, the inclusion of the communication electronics may also allow for the therapy unit 110 to be easily swapped out for replacement units, such as a therapy unit that has a fully-charged power supply. For example, by allowing a substantial portion of the patient data and associated treatment protocol data to be processed and stored by a software application on the mobile telecommunications device 112 or on a database in a remote server, substituting a different therapy unit 110 may not require a full set-up in order to configure parameters required for the treatment protocol of the specific patient. This patient-specific data and treatment protocol information may be directly synced from the software application on the mobile telecommunications device 112 to the replacement therapy unit 110 once linked to the mobile telecommunications device 112 using the communications module of the replacement therapy unit 110. Therefore, as a result of the transfer of a substantial portion of the unit control to the mobile telecommunications device 112, such as a smartphone, a therapy unit 110 with low-battery indication may be seamlessly exchanged for a fully-charged replacement therapy unit 110. The convenience of this streamlined exchange procedure may offer significant benefits over time, as in some cases the therapy unit 110 may be exchanged for a replacement unit approximately every 7 days. Notably, the patient would not be required to be tethered to a power source at any time during treatment with the therapy system 102.

The therapy unit 110 may be of a low-profile and semi-conformable, thus enabling it to be flexible to contours so that it may be affixed and worn on a patient's skin. In some embodiments, the attachment of the therapy unit 110 to the skin of the patient may be through the means of a soft, conformable, and skin-friendly adhesive. For example, in some preferred embodiments, the therapy unit 110 may be attached using a disposable silicone adhesive layer. The therapy unit 110 may be provided with multiple adhesive layers, so that these may be replaced periodically, or as needed. Various sizes and adhesive-strengths may be offered for different replacement adhesive layers, to allow a patient to modify the fit of the therapy unit 110 as needed. In some embodiments, the therapy unit 110 may also be configured with an integrated inductive coil, which may facilitate wireless charging of batteries of the therapy unit 110 while being worn, during periods of patient rest, or both.

Once the therapy unit 110 is affixed to the patient and activated, further interaction with the therapy unit 110 may be via the mobile telecommunications device 112, thus meaning that the device may be worn underneath clothing or other garments, allowing a patient to wear the necessary components of the therapy system 102 discretely with no outward signs that he or she is undergoing therapy. Accordingly, in some embodiments, all alarms and status data may be transmitted to the paired mobile telecommunications device 112, which means that the patient may not need access to the therapy unit 110, while still maintaining control over the therapy system 102 through the mobile telecommunications device 112. The software application on the mobile telecommunications device 112 may be further enabled to shut-down the therapy unit 110 in the event of a gross leak when the patient is ambulating or in public, as the dressing 108 may continue to safely absorb fluids for a period of time until the patient is able to return home or to another location where the dressing 108 or other component(s) of the therapy system 102 may be adjusted.

Additionally, some features of the therapy unit 110 may allow for the dressing 108 and low-profile conduit 272 to be used in a potentially larger range of wound treatment applications than comparably-sized systems. For example, manual pumps may require the system to be sealed to a leak-rate allowance of 0.08 ml/min. For some wound treatment applications, such sealing requirements may not be practical. However, in some embodiments, the therapy system 102 may be capable of maintaining leak rates of approximately 1 ml/min for up to 3 days, or 0.5 ml/min for 6-7 days. Furthermore, in some embodiments, the therapy unit 110 may be charged or replaced to further increase the leak capacity.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 108, the therapy unit 110, or both may be eliminated or separated from other components for manufacture or sale.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

What is claimed is:

1. An apparatus for treating a tissue site, comprising:
a therapy unit comprising:
a pneumatic pump,
a first processor operatively coupled to the pneumatic pump, the first processor configured to receive data from the pneumatic pump and generate operational data of the pneumatic pump, and
a first transceiver operatively coupled to the first processor, the first processor and the first transceiver configured to transmit the operational data of the pneumatic pump to a remote device;
wherein the remote device comprises a second transceiver configured to receive the operational data transmitted by the first transceiver, and a third transceiver configured to transmit the operational data to a monitoring center.

2. The apparatus of claim 1, wherein the fluid conduit comprises a flexible, non-woven material sealed between a plurality of occlusive layers. further comprising:
an absorbent wound dressing fluidly coupled to the therapy unit by a fluid conduit,
wherein the fluid conduit comprises a flexible, non-woven material sealed between a plurality of occlusive layers.

3. The apparatus of claim 2, wherein the plurality of occlusive layers comprises polyurethane.

4. The apparatus of claim 2, further comprising a connection interface configured to fluidly connect the fluid conduit to the absorbent wound dressing.

5. The apparatus of claim 2, wherein the fluid conduit comprises a low-profile conduit comprising a receiving end and a transmitting end separated by a length, the low-profile conduit further comprising:
a manifold comprising a plurality of fibers defining a plurality of fluid communication voids through the manifold, and
a sealing member encapsulating the manifold between the receiving end and the transmitting end.

6. The apparatus of claim 5, wherein the plurality of fibers are adapted to engage one another when the low-profile conduit is exposed to a force, and wherein the fluid communication voids are adapted to provide fluid communication through the low-profile conduit when exposed to the force.

7. The apparatus of claim 5, further comprising a conduit interface adapted to be fluidly coupled to the receiving end of the low-profile conduit.

8. The apparatus of claim 5, wherein the plurality of fibers comprises a plurality of longitudinal fibers oriented substantially in a longitudinal direction along the length of the low-profile conduit.

9. The apparatus of claim 5, wherein the plurality of fibers comprises a plurality of vertical fibers oriented substantially normal relative to the length of the low-profile conduit.

10. The apparatus of claim 5, wherein the plurality of fibers comprises:
a plurality of longitudinal fibers oriented substantially in a longitudinal direction along the length of the low-profile conduit; and
a plurality of vertical fibers oriented substantially normal relative to the longitudinal fibers;
wherein the longitudinal fibers and the vertical fibers are adapted to engage one another when the low-profile conduit is exposed to a force, and wherein the plurality of fluid communication voids are adapted to provide fluid communication through the low-profile conduit when exposed to the force.

11. The apparatus of claim 5, wherein the length of the fluid conduit does not include a tube.

12. The apparatus of claim 5, wherein the sealing member further comprises a first sealing layer and a second sealing layer, wherein the second sealing layer includes an adhesive layer on an external, tissue-facing surface.

13. The apparatus of claim 1, wherein the absorbent wound dressing comprises:
a tissue interface adapted to be placed proximate to the tissue site; and
a cover adapted to be placed over the tissue interface.

14. The apparatus of claim 13, wherein the absorbent wound dressing further comprises an absorbent layer having a superabsorbent material.

15. The apparatus of claim 1, wherein the therapy unit further comprises a battery supply, wherein the first processor and the first transceiver are configured to transmit a status of the battery supply, wherein the second processor and the second transceiver are configured to receive the status of the battery supply, and wherein the second processor and the display screen are configured to display an alert on the display screen in response to the status of the battery supply indicating a charge level of the battery decreasing below a threshold.

16. The apparatus of claim 1, wherein the remote device further comprises a user input, wherein the second processor and the second transceiver are configured to transmit a stop signal in response to a command received from the user input, wherein the first transceiver is configured to receive the stop signal, and wherein the first processor is configured to deactivate the pneumatic pump in response to the first transceiver receiving the stop signal.

17. The apparatus of claim 1, wherein the second processor and the display screen are configured to display an alert related to a fluid flow indicated by the operational data.

18. The apparatus of claim 1, wherein the therapy unit further comprises an integrated inductive coil adapted to provide a charge to a power supply of the therapy unit.

19. A method for treating a tissue site, comprising:
applying an absorbent dressing to the tissue site;
fluidly connecting the absorbent dressing to a therapy unit comprising:
a pneumatic pump,
a first processor operatively coupled to the pneumatic pump, and
a first transceiver operatively coupled to the first processor;
activating the therapy unit to provide a reduced pressure to the absorbent dressing;
receiving, at the first processor, operational data from the pneumatic pump;
generating, at the first processor, usage data of the pneumatic pump;
transmitting, from the first transceiver, usage data of the pneumatic pump;
receiving, at a second transceiver of a remote electronic device, the transmitted usage data of the therapy unit;
transmitting, from a third transceiver of the remote electronic device, the transmitted usage data of the therapy unit; and
receiving, at a monitoring center, the transmitted usage data of the therapy unit.

20. The method of claim 19, further comprising providing one or more user inputs to the remote electronic device and transmitting the one or more user inputs to the therapy unit to remotely control the therapy unit.

21. The method of claim 19, further comprising transmitting the usage data from the remote electronic device through a communications network to a remote monitoring apparatus.

22. The method of claim 21, further comprising remotely troubleshooting one or more identified issues with the therapy unit or absorbent dressing based on the transmitted usage data.

23. The method of claim 19, further comprising exchanging the therapy unit with a replacement therapy unit.

24. The method of claim 23, wherein the therapy unit is exchanged with the replacement therapy unit at an interval of approximately 3-10 days.

25. The method of claim 23, wherein the therapy unit is exchanged with the replacement therapy unit at an interval of approximately 6-8 days.

26. The method of claim 23, wherein the therapy unit is exchanged with the replacement therapy unit following a low battery status indication on the therapy unit.

27. The method of claim 23, wherein the therapy unit is exchanged without removing the absorbent dressing.

28. A therapy unit for treating a tissue site, comprising:
a negative-pressure source; and
a processor operatively coupled to the negative-pressure source, the processor configured to:
receive input information related to delivery of negative pressure from the negative-pressure source to the tissue site,
generate output information related to delivery of negative pressure from the negative-pressure source to the tissue site, and
transmit the output information from a first transceiver of the therapy unit to a mobile device via a first network, the first network adapted to allow communications between the first transceiver of the therapy unit and a second transceiver of the mobile device;
wherein the mobile device adapted to:
receive, at the second transceiver of the mobile device, the output information,
display information related to the delivery of negative pressure from the negative-pressure source to the tissue site,
collect instructions from a user related to operational parameters of the therapy unit, and
transmit the output information from a third transceiver of the mobile device to a monitoring center via a second network, the second network adapted to allow communications between the third transceiver of the mobile device and the monitoring center.

29. The therapy unit of claim 28, wherein the input information comprises the instructions from the user communicated from the mobile device.
